(19) United States
(12) Patent Application Publication
(10) Pub. No.: US 2024/0050184 A1
(43) Pub. Date: Feb. 15, 2024

(54) APPARATUS AND METHOD FOR GUIDING AN INSTRUMENT

(71) Applicant: INVIVO MEDICAL PTE. LTD., Singapore (SG)

(72) Inventors: Guohua ZENG, Guangdong (CN); Qing Hui WU, Singapore (SG); Jian Ping CHAI, Singapore (SG); Kwong Thye SIOW, Singapore (SG); Teck Kheng LEE, Singapore (SG); Soo Leong FOO, Singapore (SG)

(21) Appl. No.: 17/766,260

(22) PCT Filed: Oct. 2, 2020
(86) PCT No.: PCT/SG2020/050558

§ 371 Date: Apr. 4, 2022

(30) Foreign Application Priority Data
Oct. 3, 2019 (SG) 10201909272V

(51) Int. Cl. A61B 90/00; A61B 17/34; A61B 90/11

(52) U.S. Cl. CPC A61B 90/08; A61B 17/3403; A61B 90/11; A61B 2017/3405; A61B 2090/0811

(57) ABSTRACT

An instrument holder and a method for aligning an instrument for accessing a target within a subject, comprising a first layer, a second layer, an instrument guide with an entry and an exit point, connecting between the two layers and configured to guide the instrument from the entry point to the exit point, wherein the instrument holder is transparent to an imaging system except that the first layer and second layer include markings that are visible by the imaging system.

### FIELD OF THE DISCLOSURE

The present disclosure relates broadly to an apparatus and method for guiding an instrument for insertion into the body of a subject.

### BACKGROUND OF THE DISCLOSURE

With the use of current technologies, minimally invasive techniques/procedures are desired for accessing parts of subjects. For example, percutaneous access to the tissue such as a minimally invasive procedure to insert a tool into the body of the subject and remove tissue or to perform any type of procedure. Some procedures typically use a hollow needle to puncture the skin of a subject, and the needle is inserted in a substantially precise manner into the subject to collide with the target. In some procedures, a guide wire is inserted through the hollow needle, and the wire guides the insertion of endoscopic surgical instruments that can fragment and remove tissue. Similarly, other types of procedures may require the insertion of a tool into the body of a subject. Such procedures can include biopsies, interrogation, vertebroplasty, discectomy, and other needle or tool puncturing and intervention. Typically, techniques, such as those described above, are aided using imaging systems (e.g. x-ray, ultrasound, computerized tomography (CT) scan, magnetic resonance imaging (MRI)). For example, some processes typically use a C-arm fluoroscopy throughout the process to locate the target.

While it has been recognized that minimally invasive techniques offer benefits such as less trauma, less pain, smaller scars, less risk of infection and faster recovery, as compared to open incisions, it has also been recognized that such invasive techniques are typically difficult to perform. Currently, the insertion process of a tool into the body of a subject is significantly dependent on the experience of the surgeon to manipulate and align the tool. It is recognized that the tedious and time-consuming part of the manual procedure is the alignment and manipulation of the tool or the needle.

Furthermore, prolonged procedural time during usage of minimally invasive techniques typically increases the risk of perioperative complications of subjects. In addition, prolonged procedural time typically increases the health risk of surgical crews due to exposure to radioactive imaging. Furthermore, it has been recognized that prolonged procedural time imposes a burden on hospitals given that operating theatres are, typically, in constant demand.

In view of the challenges facing techniques such as the current procedures described above, robotic systems are being developed to facilitate or assist in such procedures. For example, a number of automated robotic systems aided by image guiding system and motorized needle insertion driver is currently in development. Robotic systems also encounter some of the limitations that current practitioners face when guiding an instrument into the body of a subject.

U.S. Patent Publication 2016/0206383 by the current applicant and published Jul. 21, 2016, the disclosure of which is incorporated herein by reference describes a system and apparatus for guiding an instrument.

It is desirable to provide an improved apparatus and method given the current system used by a practitioner for guiding an instrument into the body of a subject.

### SUMMARY OF THE DISCLOSURE

An aspect of an embodiment of the disclosure, relates to an instrument holder for performing a process of aligning an instrument (e.g. a needle, hollow needle, tool, wire) used to access a target (e.g. a tumor, kidney stone, abscess, or other item) within a subject. The instrument holder may be any device that holds an instrument.

The instrument holder is configured to be held by a rigid arm or an articulated arm and be positioned so that the instrument can be inserted into the subject to hit the target.

The instrument holder includes a first layer with an entry point for inserting the instrument and a second layer with an exit point for the instrument to exit from the holder. Both the entry point and the exit point may also be located within the second layer.

The instrument includes a guiding channel to guide the instrument connecting between the entry point to the exit point.

The process is performed with the aid of an imaging system (e.g. a C-arm, ultrasound, CT scan, MRI), which continuously displays on a display the instrument and the target. Alternatively, the imaging system (e.g. x-ray machine) can display snapshots of the instrument and the target on a display. The first layer and second layer of the instrument holder are equipped with markings that can be seen on the display of the imaging system during the process. The markings can cover the entire layer. The markings on the first layer are designed to form a predefined pattern with the markings of the second layer when aligned so that the instrument will collide with the target when inserted into the subject. Alternatively, an instrument inserted into the second layer can serve as markings on the second layer to align with the markings on the first layer.

The disclosure can be applied to any method for referencing a position of an instrument to be used in conjunction with a medical imaging device.

There is thus provided according to an exemplary embodiment of the disclosure, an instrument holder for aligning an instrument for accessing a target within a subject, comprising: a first layer; a second layer; an instrument guide having an entry point and an exit point, the instrument guide connecting between the first layer and the second layer and configured to guide the instrument from the entry point to the exit point; wherein the first layer and the second layer include first layer markings and second layer markings that are visible by an imaging system; and wherein the first layer markings are designed to forma predefined pattern with the second layer markings relative to the target as viewed on a display of the imaging system, when aligning the first layer and the second layer so that the instrument will collide with the target.

In an exemplary embodiment of the disclosure, the first layer markings include a first geometric shape and the second layer markings include a second geometric shape distinct from the first geometric shape. Optionally, the first geometric shape comprises a line and the second geometric shape comprises one or more pairs of arrows or arrow heads, and in the predefined pattern the one or more pairs of arrows or arrow heads point at the line, which is situated between the one or more pairs of arrows or arrow heads. Optionally, in the predefined pattern, there is a gap between the one or more pairs of arrows or arrow heads and the line. Alternatively, the first geometric shape comprises lines representing linear graduations of a unit of measure and the second geometric shape comprises a line, and in the predefined pattern the line of the second geometric shape aligns at a middle line of the first geometric shape. Alternatively, the first geometric shape comprises one or more pairs of arrows or arrowheads and the second geometric shape comprises two lines in the shape of a cross with the exit point at the center, and in the predefined pattern the one or more pairs of arrows or arrow heads point at the lines of the second geometric shape such that each line is situated between the one or more pairs of arrows or arrow heads. Optionally, in the predefined pattern, there is a gap between the one or more pairs of arrows or arrow heads and the lines of the second geometric shape. Alternatively, the first geometric shape comprises two or more sets of lines representing linear graduations of a unit of measure and the second geometric shape comprises two lines in the shape of a cross with the exit point at the center, and in the predefined pattern each of the lines of the second geometric shape aligns with a middle line of each of the sets of lines representing linear graduations of a unit of measure. Alternatively, the first layer markings include a cross with the entry point at the center and the second layer markings include a cross with the exit point at the center, and in the predefined pattern the cross of the first layer marking and the cross of the second layer marking coincide. Optionally, the instrument holder further comprises a protuberance extending from the second layer.

In an exemplary embodiment of the disclosure, the instrument is a hollow needle. Alternatively, the instrument is an elongated tool. In an exemplary embodiment of the disclosure, the instrument holder includes handles to fit a positioning arm. Optionally, the handles are configured to enable the instrument holder to rotate relative to the positioning arm. In an exemplary embodiment of the disclosure, the first layer or the second layer include additional markings, which are not part of the predefined pattern to identify the first layer or the second layer by a user when aligning. Optionally, the target is located percutaneously. In an exemplary embodiment of the disclosure, the instrument holder is transparent to the imaging system. Optionally, the instrument guide is perpendicular to the first layer and the second layer. In an exemplary embodiment of the disclosure, the instrument guide forms a preselected angle with the first layer and with the second layer.

There is further provided according to an exemplary embodiment of the disclosure, a method of aligning an instrument to a target within a subject, comprising: providing an instrument holder as described above on a positioning arm; positioning an imaging system to show on a display the target and the instrument holder, and aligning the instrument holder so that the first layer markings and the second layer markings form a predefined pattern relative to the target on the display of the imaging system.

In an exemplary embodiment of the disclosure, the instrument is being aligned for percutaneous access to the target within the subject. Optionally, the positioning arm is an articulated arm positioned above the subject.

There is further provided according to an exemplary embodiment of the disclosure, an instrument holder for aligning an instrument for accessing a target within a subject, comprising: a first layer; a second layer, the second layer connected to the first layer by a column; an instrument guide having an entry point and an exit point, the instrument guide located within the second layer and configured to guide the instrument from the entry point to the exit point; wherein the first layer includes markings forming a first layer markings that are visible by an imaging system; and wherein the first layer markings are designed to form a predefined pattern with the instrument visible by the imaging system within the second layer relative to the target as viewed on a display of the imaging system, when aligning the first layer and the second layer so that the instrument will collide with the target.

In an exemplary embodiment of the disclosure, the first layer markings include a first geometric shape. Optionally, the first geometric shape comprises lines representing linear graduations of a unit of measure and in the predefined pattern the instrument aligns at a middle line of the first geometric shape. Alternatively, the first geometric shape comprises one or more pairs of arrows or arrow heads, and in the predefined pattern the one or more pairs of arrows or arrow heads point at the instrument, which is situated between the one or more pairs of arrows or arrow heads. Optionally, in the predefined pattern, there is a gap between the one or more pairs of arrows or arrow heads and the instrument. Optionally, the instrument holder further comprises a protuberance extending from the second layer.

In an exemplary embodiment of the disclosure, the instrument is a hollow needle. Alternatively, the instrument is an elongated tool. In an exemplary embodiment of the disclosure, the instrument holder includes handles to fit a positioning arm. Optionally, the handles are configured to enable the instrument holder to rotate relative to the positioning arm. In an exemplary embodiment of the disclosure, the first layer or the second layer include additional markings, which are not part of the predefined pattern to identify the first layer or the second layer by a user when aligning. Optionally, the target is located percutaneously. In an exemplary embodiment of the disclosure, the instrument holder is transparent to the imaging system.

There is further provided according to an exemplary embodiment of the disclosure a method of aligning an instrument to a target within a subject, comprising: providing an instrument holder as described above on a positioning arm; inserting the instrument into the instrument holder; positioning an imaging system to show on a display the target, the instrument holder and the instrument; and aligning the instrument holder so that the first layer markings and the instrument form a predefined pattern relative to the target on the display of the imaging system.

In an exemplary embodiment of the disclosure, the instrument is being aligned for percutaneous access to the target within the subject. Optionally, the positioning arm is an articulated arm positioned above the subject.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1A is a bottom perspective view of an instrument holder, according to an exemplary embodiment of the disclosure;

FIG. 1B is a top perspective view of an instrument holder, according to an exemplary embodiment of the disclosure;

FIG. 2A is a bottom perspective view of a first alternative instrument holder, according to an exemplary embodiment of the disclosure;

FIG. 2B is a top perspective view of a first alternative instrument holder, according to an exemplary embodiment of the disclosure;

FIG. 2C is a top view of a first alternative instrument holder, according to an exemplary embodiment of the disclosure:

FIG. 3A is a side perspective view of a second alternative instrument holder, according to an exemplary embodiment of the disclosure;

FIG. 3B is a bottom view of a second alternative instrument holder, according to an exemplary embodiment of the disclosure;

FIGS. 3C-3D are illustrations of a first examples of markings on the second alternative instrument holder, and FIG. 3E is an illustration of a predefined pattern formed by exemplary markings on the second alternative instrument holder, according to an exemplary embodiment of the disclosure;

FIGS. 3F-3G are illustrations of a second other examples of markings on the second alternative instrument holder, and FIG. 3H is an illustration of a predefined pattern formed by exemplary markings on the second alternative instrument holder, according to an exemplary embodiment of the disclosure;

FIG. 4A is a bottom perspective view of a third alternative instrument holder, according to an exemplary embodiment of the disclosure;

FIG. 4B is a top perspective view of a third alternative instrument holder, according to an exemplary embodiment of the disclosure;

FIG. 4C is a top view of a third alternative instrument holder, according to an exemplary embodiment of the disclosure;

FIGS. 5A to 5G are schematic illustrations of a process of aligning an instrument holder, according to an exemplary embodiment of the disclosure;

FIGS. 6A to 6F are schematic illustrations of a first alternative process of aligning an instrument holder, according to another exemplary embodiment of the disclosure; and

FIGS. 7A to 7D are schematic illustrations of a second alternative process of aligning an instrument holder, according to an exemplary embodiment of the disclosure.

With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

Identical or duplicate or equivalent or similar structures, elements, or parts that appear in one or more drawings are generally labeled with the same reference numeral, optionally with an additional letter or letters to distinguish between similar entities or variants of entities, and may not be repeatedly labeled and/or described. References to previously presented elements are implied without necessarily further citing the drawing or description in which they appear.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

### DETAILED DESCRIPTION

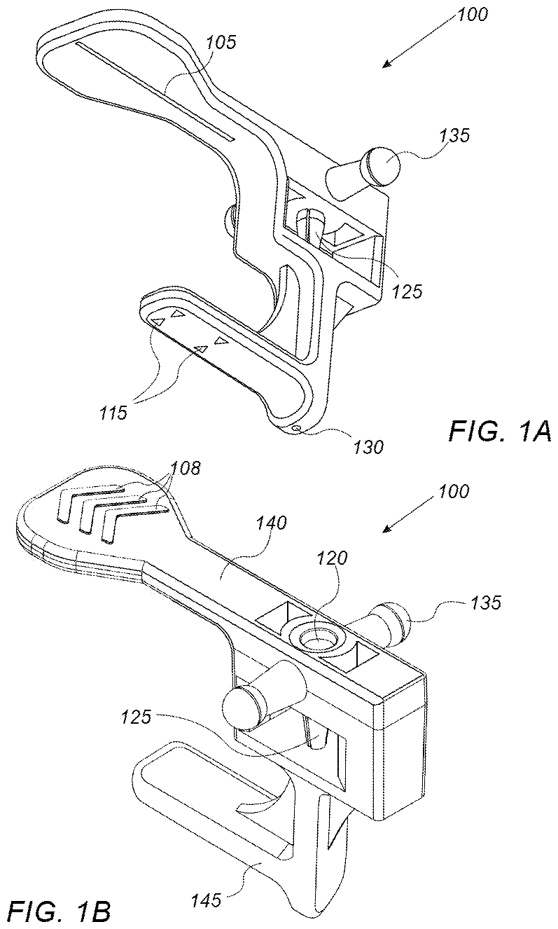

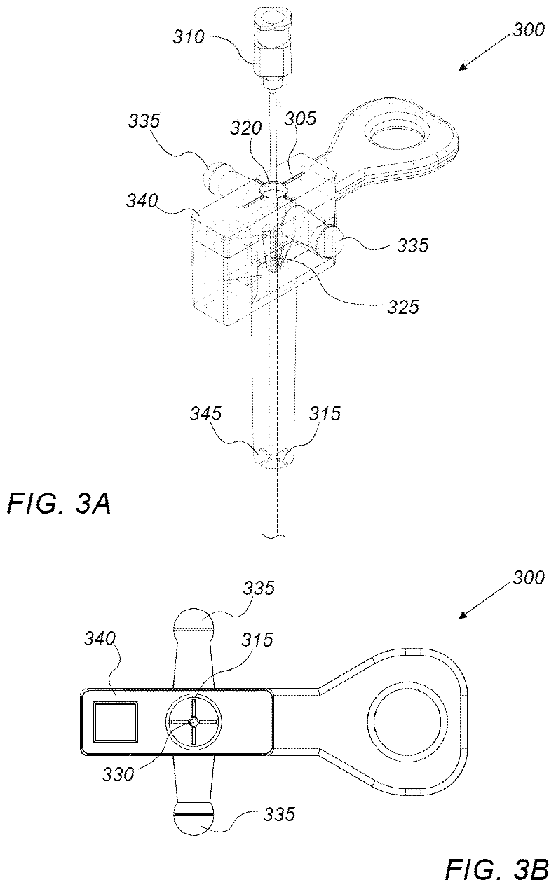

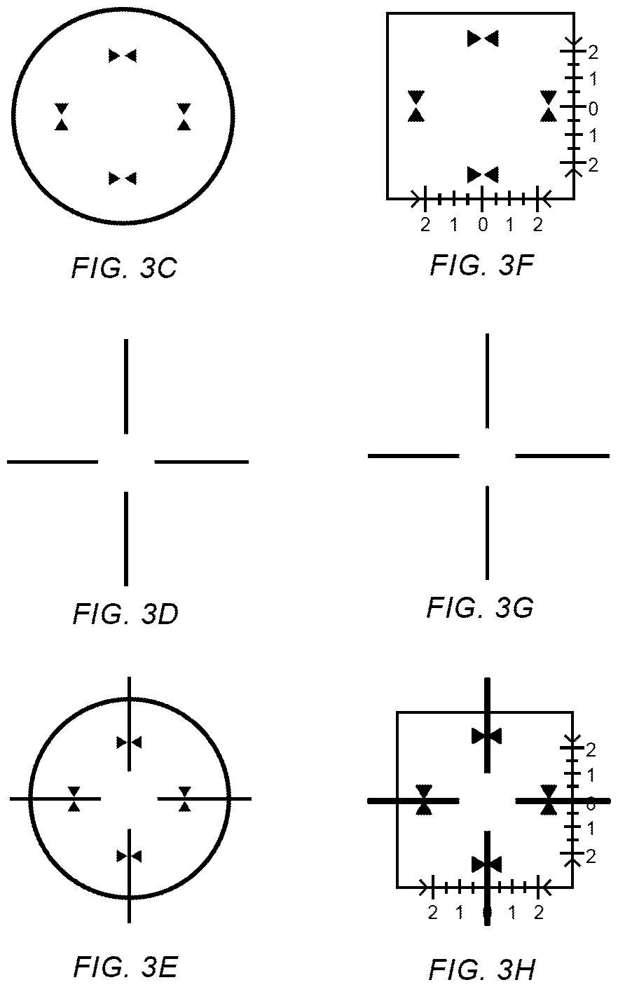

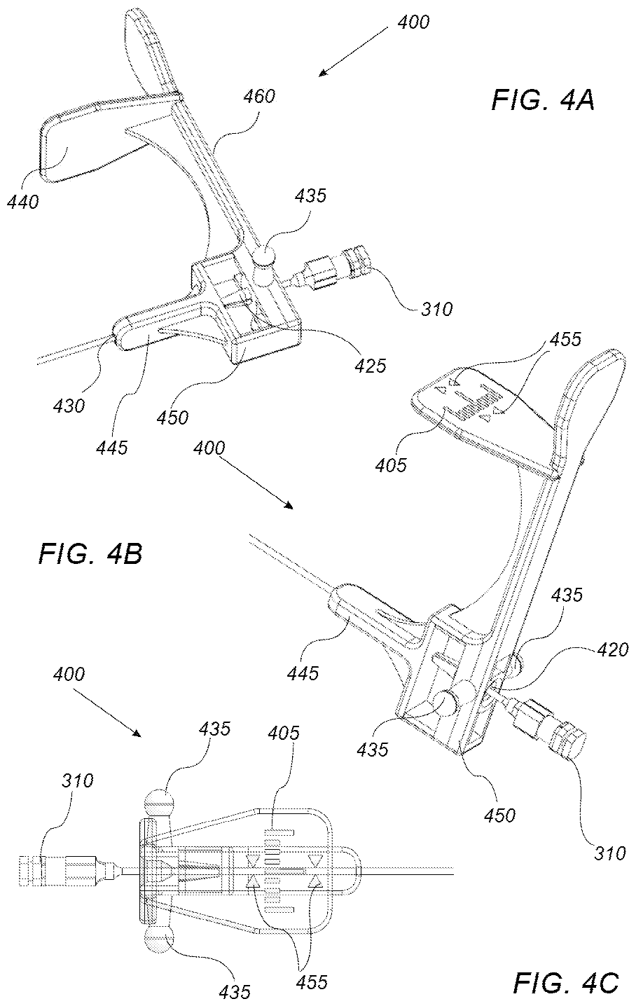

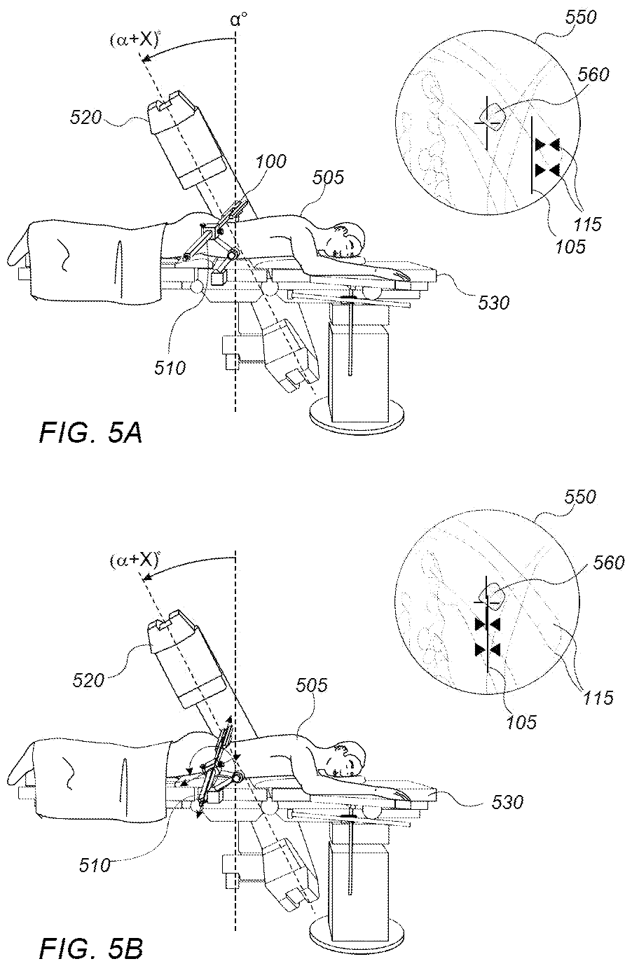

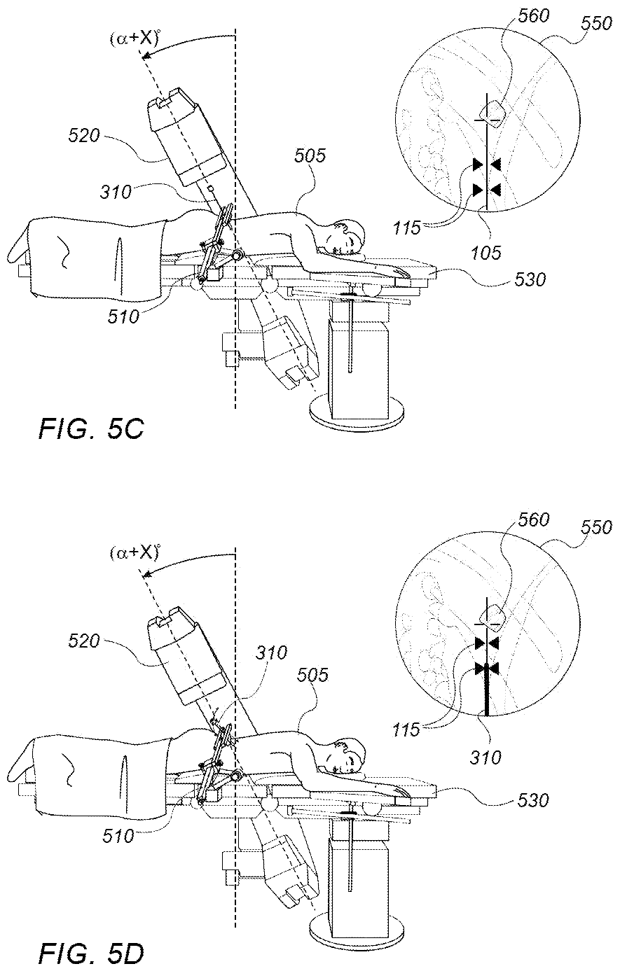

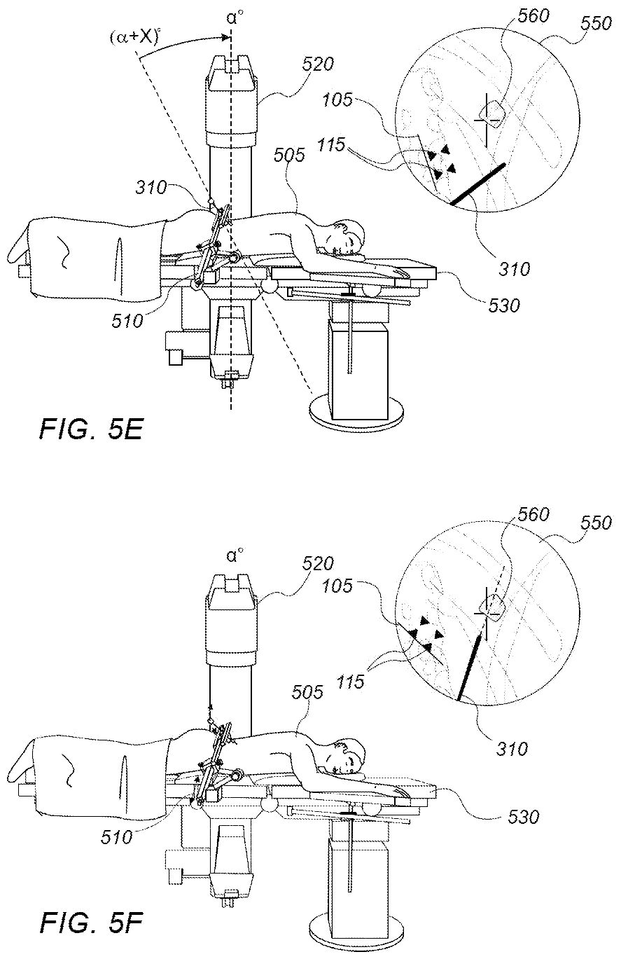

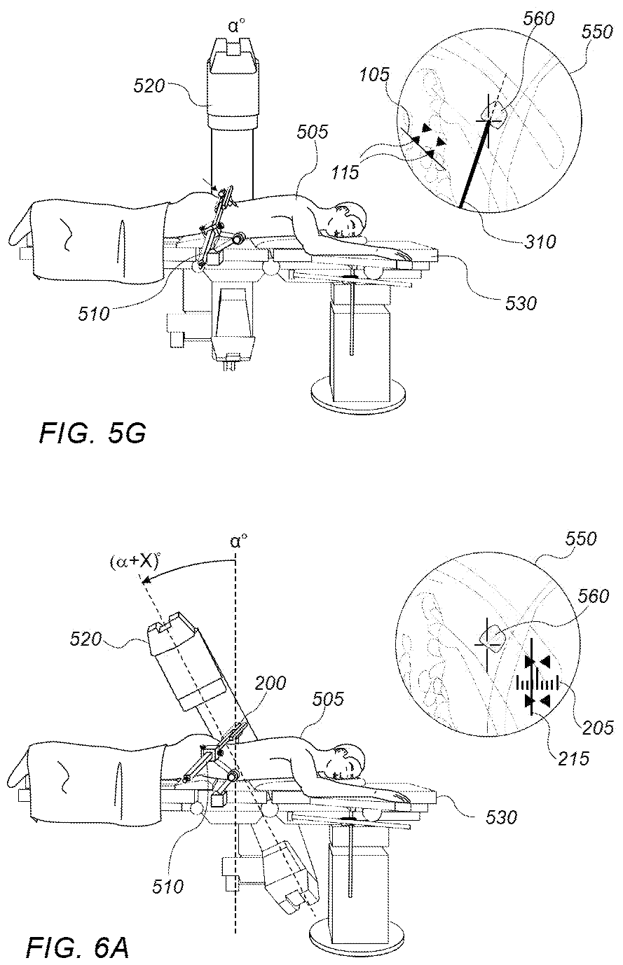

FIG. 1A is a bottom perspective view of an instrument holder 100, and FIG. 1B is a top perspective view of an instrument holder 100, according to an exemplary embodiment of the disclosure. Instrument holder 100 serves to hold an instrument 310 (e.g. as shown in FIGS. 3A and 4A to 4C) for percutaneous access to the body of a subject. In an exemplary embodiment of the disclosure, a positioning arm 510 (e.g. a rigid arm or an articulated arm as shown in FIGS. 5A to 5G, 6A to 6F and 7A to 7D) supports instrument holder 100 and is used to lock the instrument holder 100 in a specific position and orientation above the subject, so that instrument 310 (e.g. a hollow needle or other type of probe) may be inserted into the instrument holder and guided accurately into the body of the subject. Optionally, instrument holder 100 includes handles 135 to fit the positioning arm 510. The handles 135 are held by the positioning arm 510 to hold the instrument holder 100 stable or to be rotated relative to the positioning arm 510. The handles 135 may be pairs of spherical protrusions connected to the instrument holder 100 at any pat of the body of the instrument holder 100, including along the first layer 140 or second layer 145. Additionally, the instrument holder 100 includes three dimensional markings for assisting in aligning the instrument 310 for percutaneous access to a target. It will be understood that any number of markings may be used to form the three-dimensional markings.

In an exemplary embodiment of the disclosure, positioning the instrument holder 100 is performed under an imaging system 520 (FIGS. 5A to 5G, 6A to 6F and 7A to 7D) (e.g. an X-ray or C-arm) that can see through the instrument holder 100 and the body of the subject to align the instrument 310 to be pointed at a desired point within the body of the subject, for example aligning a hollow needle to reach a stone within the subject's kidney. Optionally, the instrument 310 is not transparent to the imaging system 520, so that it is viewed on a display of the imaging system 520. For example, the instrument holder 100 may be made from plastic so that it is transparent and the instrument 310 may be made from metal to be non-transparent to the imaging system 520.

In an exemplary embodiment of the disclosure, the instrument holder 100 includes a first layer 140 and a second layer 145, which are connected together by an instrument guide 125 that is configured to hold the instrument 310 for access to the subject. The instrument guide is also adapted to prevent bending of the instrument 310 when penetrating the skin of a patent. Optionally, the instrument guide 125 is perpendicular to the two layers (140, 145) or forms a preselected angle.

In an exemplary embodiment of the disclosure, the first layer 140 includes an entry point 120 for inserting the instrument 310 to the first layer 140 and a first layer marking 105 (or marks) that are non-transparent to the imaging system 520 to identify the first layer 140 and align it relative to the second layer 145. Optionally, the first layer marking 105 may be a line. In some embodiments of the disclosure, the first layer 140, the second layer 145 and the instrument guide 125 are formed as a single mold. In some embodiments of the disclosure, the instrument guide 125, entry point 120 and the corresponding exit point 130 may all be located in a single plane, and not necessarily between the handles 135.

In an exemplary embodiment of the disclosure, the second layer 145 includes an exit point 130 from which the instrument 310 exits the instrument holder 100, and includes one or more second layer markings 115 that are non-transparent to the imaging system 520 to enable the imaging system 520 to identify the second layer 145 and enable the user to align the second layer 145 relative to the first layer 140. Optionally, the second layer markings 115 comprise one or two pairs of arrows. Optionally, the one or two pairs of arrows are triangular and are aligned such that their respective vertices are facing one another. Optionally, instrument guide 125 guides the instrument 310 from the entry point 120 to the exit point 130 through the body of the instrument holder 100 and optionally perpendicular to the first layer 140 and second layer 145, or at a preselected angle.

In an exemplary embodiment of the disclosure, the first layer markings 105 are designed to form a predefined pattern with the second layer markings 115 as viewed on a display 550 (e.g. see FIGS. 5A and 5B) of the imaging system 520 near the target 560, when aligning the first layer 140 and the second layer 145 so that the instrument will collide with the target 560 when inserted into the subject. Persons skilled in the art will appreciate that the instrument may alternatively penetrate, enter, touch, stab, impale or otherwise interact with the target in such a manner as required for the relevant procedure by the practitioner.

In some embodiments of the disclosure, additional transparent markings 108 that are not part of the predefined pattern, are placed on the first layer 140 or the second layer 145 to enable a user of the instrument holder 100 to better grip the instrument holder 100 and/or view the instrument holder 100.

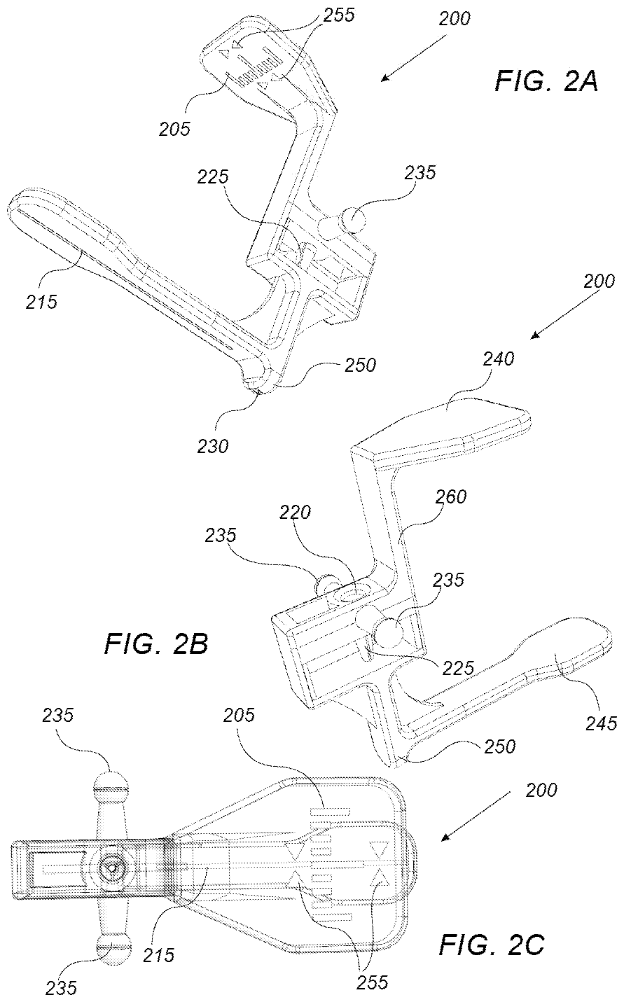

FIG. 2A is a bottom perspective view of a first alternative instrument holder 200, FIG. 2B is a top perspective view of a first alternative instrument holder 200, and FIG. 2C is a top view of a first alternative instrument holder 200, according to an exemplary embodiment of the disclosure. As in instrument holder 100, instrument holder 200 comprises handles 235 for holding the instrument holder 200 by positioning arm 510 or rotating the instrument holder 200 relative to the positioning arm 510. The handles 235 may be pairs of spherical protrusions form part of or are connected to the instrument holder 200 at any part of the body of the instrument holder 200, including along the first layer 240 or second layer 245. The instrument holder 200 also includes a first layer 240 and a second layer 245, which are connected by an instrument guide 225 that is configured to hold the instrument 310 for access to the subject. Optionally, the instrument guide 225 is perpendicular to the two layers (240, 245) or forms a preselected angle.

In an exemplary embodiment of the disclosure, the body 260 of instrument holder 200 which connects the first layer 240 and the second layer 245 includes an entry point 220 for inserting instrument 310 into instrument holder 200. Preferably, the distance between the first layer 240 and the second layer 245 is between 5 to 10 centimeters, and ideally around 6 centimeters.

In an exemplary embodiment of the disclosure, the first layer 240 includes a first layer marking 205 in the form of lines representing linear graduations of a unit of measure (e.g. centimeter or inch). Preferably, the distance between the lines of the first layer marking 205 is 2 millimeters, with the entire first layer marking 205 marking an overall distance of 16 millimeters. Persons skilled in the art will appreciate that the distance between the lines of the first layer marking 205 may be any distance as long as the imaging system can accurately differentiate the lines, and that the distance between the lines of the first layer marking 205 should change proportionally in relation to the distance between the first layer 240 and the second layer 245. The first layer marking 205 (or markings) are non-transparent to the imaging system to identify the first layer 240 and align it relative to the second layer 245. Optionally, the first layer marking 205 includes one or more pairs of arrows 255. Optionally, the one or two pairs of arrows 255 are triangular and are aligned such that their respective vertices are facing one another. Optionally, the one or two pairs of arrows 255 are situated above or below the middle line of the lines representing linear graduations of a unit of measure such that the gap between the vertices of the one or more pairs of arrows 255 align with the middle line of the lines representing linear graduations of a unit of measure.

In an exemplary embodiment of the disclosure, the second layer 245 includes an exit point 230 from which the instrument 310 exits instrument holder 200, and includes a second layer marking 215 that is non-transparent to the imaging system 520 to enable imaging system 520 to identify the second layer 245 and enable the user to align it relative to the first layer 240. Preferably, the second layer marking 215 is in the shape of a line. The first layer marking 205 and second layer marking 215 are formed from a material, such as a radio-opaque material that can be seen on the display of the imaging system 520 (e.g. a metallic material).

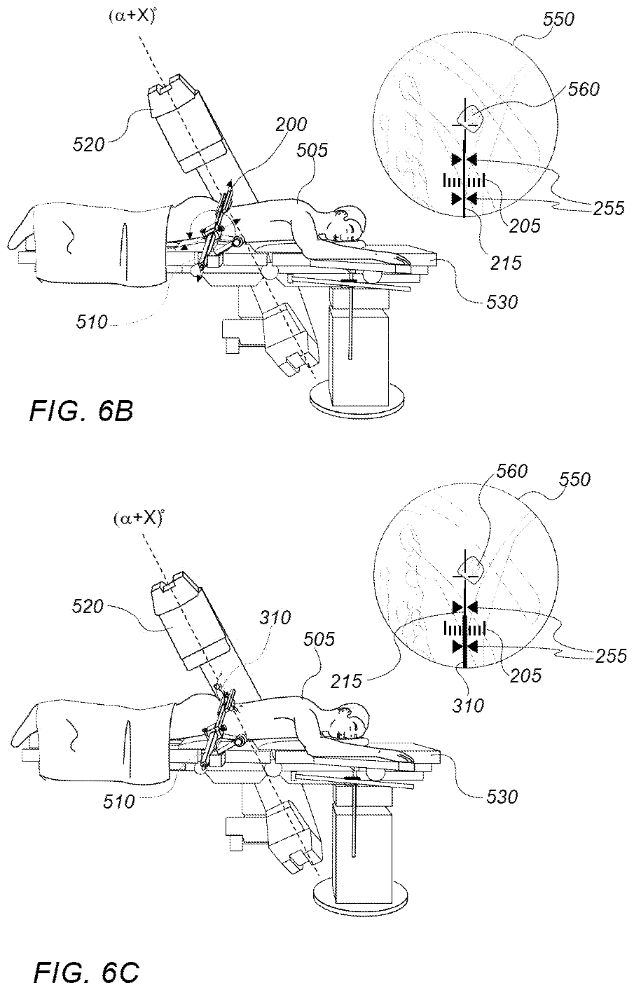

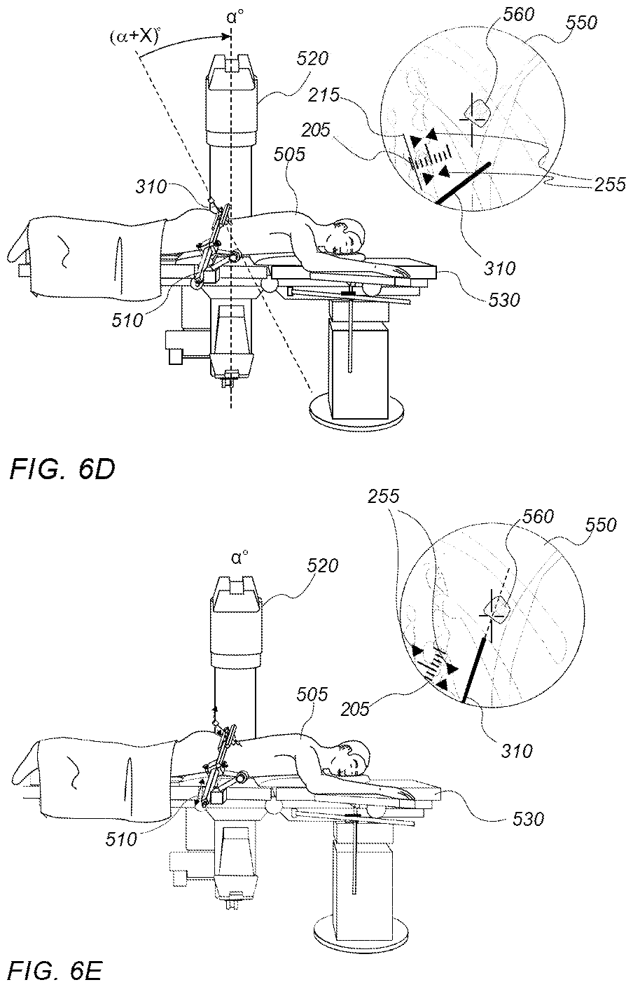

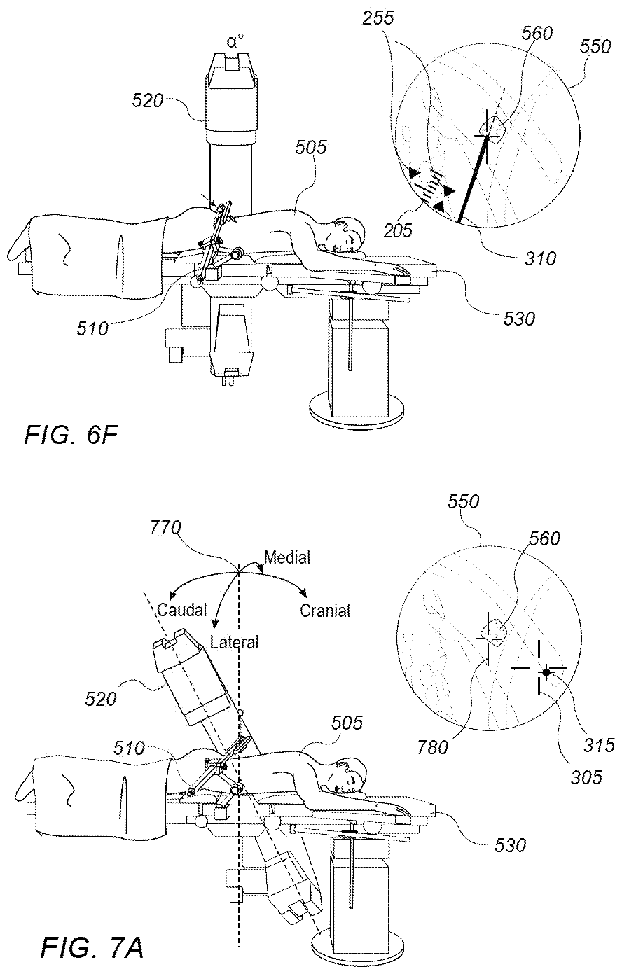

In an exemplary embodiment of the disclosure, the first layer marking 205 is designed to form a predefined pattern with the second layer marking 215 as viewed on a display 550 (see FIG. 6C) of the imaging system 520 near the target when aligning the first layer 240 and the second layer 245 so that the instrument 310 will collide with the target 560 when inserted into the subject. In the predefined pattern, the second layer marking 215 will align at the middle line of the first layer marking 205 (see FIG. 2C). Preferably, the line comprising the second layer marking 215 is of a different thickness from the lines comprising the first layer marking 205 to assist the user in differentiating the lines comprising the first layer marking 205 and the second layer marking 215. Preferably, the line comprising the second layer marking 215 is narrower than the lines comprising the first layer marking 205. Preferably, in the predefined pattern, the second layer marking 215 will align between the one or two pairs of arrows 255 in the first layer marking 205. Preferably, in the predefined pattern, there is a gap between the second layer marking 215 and the vertices of the one or two pairs of arrows 255 in the first layer marking 205 to assist the user in aligning the first layer 240 and the second layer 245. Preferably, in the predefined pattern, the gap between the second layer marking 215 and the vertices of the one or two pairs of arrows 255 in the first layer marking 205 is between 0.1 and 0.5 millimeters, and ideally 0.2 millimeters.

In an exemplary embodiment of the disclosure, there is a protuberance 250 extending from the second layer 245. Optionally, the protuberance 250 is perpendicular to the second layer 245 or forms a preselected angle with the second layer 245. Preferably, the protuberance 250 is located around the exit point 230 from which the instrument 310 exits the instrument holder 200. The protuberance 250 reduces the distance between the exit point 230 of the instrument holder 200 and the skin surface of a patient to prevent bending of the instrument 310 when penetrating the skin of a patient. Preferably, the protuberance 250 is rounded for safety in case of contact with the skin surface of a patient. Preferably, the protuberance 250 is between 2 to 10 millimeters, and ideally around 5 millimeters. Optionally, instrument guide 225 guides the instrument 310 from the entry point 220 to the exit point 230 through the body 260 of the instrument holder 200 and optionally perpendicular to the first layer 240 and second layer 245, or at a preselected angle.

FIG. 3A is a side perspective view of a second alternative instrument holder 300, and FIG. 3M is a bottom view of a second alternative instrument holder 300, according to an exemplary embodiment of the disclosure. As in the previous embodiments, instrument holder 300 includes handles 335 to fit the positioning arm 510. The handles 335 are for holding the instrument holder 300 by positioning arm 510 or rotating the instrument holder 300 relative to the positioning arm 510. The handles 335 may be pairs of spherical protrusions connected to the instrument holder 300 at any part of the body of the instrument holder 300, including along the first layer 340 or second layer 345. As in the previous embodiments, instrument holder 300 also includes a first layer 340 and a second layer 345, which are connected together by an instrument guide 325 that is perpendicular to the two layers (340, 345) and is configured to hold the instrument 310 for access to the subject. Optionally, the first layer 340 includes an entry point 320 for inserting instrument 310 to first layer 340. Likewise, the first layer 340 includes a first layer marking 305 in the form of a cross with entry point 320 at the center. Optionally, the first layer marking 305 may comprise one or more pairs of arrows. Optionally, the one or more pairs of arrows are triangular and are aligned such that their respective vertices are facing one another. Optionally, the first layer marking 305 may comprise one or more sets of lines representing linear graduations of a unit of measure (e.g. centimeter or inch). Optionally, the first layer markings 305 may additionally include numerals on the one or more sets of lines representing linear graduations of a unit of measure to indicate the deviation of certain lines from the middle line of the one or more sets of lines representing linear graduations of a unit of measure.

In an exemplary embodiment of the disclosure, the second layer 345 includes an exit point 330 from which the instrument 310 exits instrument holder 300 to the second layer 345, and includes second layer markings 315 in the shape of a cross with exit point 330 at the center of the cross-shaped second layer marking 315 to enable imaging system 520 to identify the second layer 345 and enable the user to align it relative to the first layer 340. The first layer marking 305 and second layer marking 315 are formed from a material, such as a radio-opaque material that can be seen on the display of the imaging system 520 (e.g. a metallic material). Optionally, instrument guide 325 guides the instrument 310 from the entry point 320 to the exit point 330 through the body of the instrument holder 300 to the first layer 340 and second layer 345. Optionally, the instrument guide 325 is perpendicular to the first layer 340 and second layer 345 or forms a preselected angle.

In an exemplary embodiment of the disclosure, the first layer markings 305 are designed to coincide with the second layer markings 315 forming a cross above the target as viewed on a display 550 of the imaging system 520 when aligning the first layer 340 and the second layer 345 so that the internment 310 will collide with the target 560 when inserted into the subject.

Alternatively, the first layer markings 305 are designed to form a predefined pattern with the second layer markings 315 as viewed on a display 550 of the imaging system 520 when aligning the first layer 340 and the second layer 345 so that the instrument 310 will collide with the target 560 when inserted into the subject. Optionally, in the predefined pattern, each of the lines of the second layer marking 315 will align between the one or more pairs of arrows of the first layer marking 305, and there is a gap between the lines of the second layer marking 315 and the tops of the arrows or arrowheads of the first layer marking 305. Optionally, in the predefined pattern, each of the lines of the second layer marking 315 will align at the middle line of each of the one or more sets of lines representing linear graduations of a unit of measure of the first layer markings 305.

FIGS. 3C-3D are illustrations of examples of markings on the second alternative instrument holder 300, and FIG. 3E is an illustration of a predefined pattern formed by exemplary markings on the second alternative instrument holder 300, according to an exemplary embodiment of the disclosure. FIG. 3C is a first example of a marking to be implemented as the first layer marking 305 of FIG. 3A, comprising four pairs of triangular arrows, each triangular arrow pair is aligned such that their respective vertices are facing one another, in a radial arrangement. FIG. 3D is a second example of a marking to be implemented as the second layer marking 315, comprising a crosshair marking. FIG. 3E shows a predefined pattern formed by the first layer making 305 and second layer marking 315 implementing the first and second examples (FIG. 3C, 3D) as viewed on a display 550 of the imaging system 520 during the process of aligning the device of the present disclosure. As can be seen, according to the first and second example, the lines of the crosshair of the second layer marking 315 align between each of the four pairs of arrows of the first layer marking 305.

FIGS. 3F-3G are illustrations of other examples of markings on the second alternative instrument holder 300, and FIG. 3H is an illustration of a predefined pattern formed by exemplary markings on the second alternative instrument holder 300, according to an exemplary embodiment of the disclosure. FIG. 3F is a third example of a marking to be implemented as the first layer marking 305 of FIG. 3A, comprising four pairs of triangular arrows, each triangular arrow pair is aligned such that their respective vertices are facing one another, in a radial arrangement and a square, with two sets of lines representing linear graduations of a unit of measure on two adjacent sides of the square, the middle line of each of the two sets of lines representing linear graduations of a unit of measure aligned between the vertices of the pairs of triangular arrows located on the at least two adjacent sides of the square. FIG. 3G is a fourth example of marking to be implemented as the second layer marking 315, comprising a crosshair marking. FIG. 3H shows a predefined pattern formed by the first layer making 305 and second layer marking 315 implementing the third and fourth examples (FIGS. 3F and 3G) as viewed on a display 550 of the imaging system 520 during the process of aligning the device of the present disclosure. As can be seen, according to the third and fourth example, the lines of the crosshair of the second layer marking 315 align between each of the four pairs of arrows of the first layer marking 305 and at the middle line of each of the two sets of lines representing linear graduations of a unit of measure of the first layer marking 305.

FIG. 4A is a bottom perspective view of a third alternative instrument holder 400, FIG. 4B is a top perspective view of a third alternative instrument holder 400, and FIG. 4C is a top view of a third alternative instrument holder 400 according to an exemplary embodiment of the disclosure. As in the previous embodiments, instrument holder 400 includes handles 435 to fit the positioning arm 510. The handles 435 are for holding the instrument holder 400 by positioning arm 510 or rotating the instrument holder 400 relative to the positioning arm 510. The handles 435 may be pairs of spherical protrusions connected to the instrument holder 400 at any part of the body of the instrument holder 400, including along the first layer 440 or second layer 445. As in the previous embodiments, instrument holder 400 also includes a first layer 440 and a second layer 445, which are connected together by a column 460. Preferably, there is a protuberance 450 extending from the second layer 445. Preferably, the protuberance 450 is rounded for safety in case of contact with the skin surface of a patient. Preferably, the protuberance 450 is between 2 to 10 millimeters, and ideally around 5 millimeters. Optionally, the protuberance 450 is perpendicular to the second layer 445 or forms a preselected angle with the second layer 445. Optionally, the column 460 is perpendicular to the two layers (440, 445). The column 460 can be of any shape, including straight or circular.

In an exemplary embodiment of the disclosure, the second layer 445 includes an entry point 420 for inserting instrument 310, an exit point 430 for the instrument 310 to exit the instrument holder 400, and an instrument guide 425 to guide an instrument 310 from entry point 420 to exit point 430. The first layer 440 includes a first layer marking 405. The first layer marking 405 is formed from a material, such as a radio-opaque material that can be seen on the display of the imaging system 520 (e.g. a metallic material). Optionally, the first layer marking 405 is in the form of lines representing linear graduations of a unit of measure (e.g. centimeter or inch). Optionally, the first layer marking 405 also includes one or more pairs of arrows 455.

In an exemplary embodiment of the disclosure, instrument 310 is formed from a material, such as a radio-opaque material that can be seen on the display of the imaging system 520 (e.g. a metallic material). Once inserted into instrument holder 400, the instrument 310 enables the imaging system to identify the second layer 445 and enables the user to align it relative to the first layer 440. Preferably, the second layer 445 is aligned relative to the first layer 440 when the instrument 310 is aligned at the middle line of the first layer marking 405. Preferably, the second layer 445 is aligned relative to the first layer 440 when the instrument 310 is aligned between the one or two pairs of arrows 455 in the first layer marking 405. Preferably, there is a gap between the instrument 310 and the vertices of the one or two pairs of arrows 455 in the first layer marking 405 when the first layer 440 is aligned with the second layer 445. Preferably, the gap between the instrument 310 and the vertices of the one or two pairs of arrows 455 in the first layer marking 405 is between 0.1 and 0.5 millimeters, and ideally 0.2 millimeters.

In an exemplary embodiment of the disclosure, the first- and second-layer markings may be vice versa and not limited to arrows and lines or crosses. They can be any kind of 2-dimensional geometric shapes including and not limited to circles/semicircles, triangles, square, rectangles, polygon, ovals, etc. so long as it can be appropriately used for alignment and matching.

Additionally, the markings of the two layers can be aligned to establish a common axis that passes through all the markings. The markings can be further aligned to the instrument and a target so that all three (markings, instrument, target) will lie along the same axis. In establishing the axis, the instrument is set to comply to move only along that axis.

FIGS. 5A to 5G are schematic illustrations of a process of aligning instrument holder 100, according to an exemplary embodiment of the disclosure. In an exemplary embodiment of the disclosure, as shown in FIG. 5A, a subject 505 is positioned on a surgical table 530 with instrument holder 100 mounted on positioning arm 510 above the subject 505. The positioning arm 510 is typically attached to the surgical table 530 and an imaging system 520 is initially positioned at an angle α relative to the vertical. With the help of imaging system 520 the practitioner views the display 550 and selects an angled plane relative to a (for example at an angle X), which will enable optimal insertion of the instrument 310 to hit the target 560 (e.g. a kidney stone). Additionally, the practitioner selects a puncture angle for tilting the instrument 310 in the selected angled plane to accurately hit the target 560.

In an exemplary embodiment of the disclosure, the practitioner rotates the imaging system 520 to the desired angle (X+α) and views instrument holder 100 as appearing on display 550.

As shown in FIG. 5B the practitioner uses the positioning arm 510 to align the first layer marking 105 of instrument holder 100 to point toward target 560 and to be in the center of second layer marking 115. Optionally, first layer marking 105 is in the form of a line and second layer marking 115 is in the form of two pairs of arrows. When instrument holder 100 is aligned with the selected plane the line of the first layer marking 105 will be centered between the arrows of the second layer marking 115.

As shown in FIG. 5C the practitioner inserts the instrument 310 (e.g. a hollow needle) into the instrument holder 100. Initially the instrument 310 is not visible since it is perpendicular to the first layer marking 105 and not aligned to pass exactly through the target.

As shown in FIG. 5D the practitioner decides on an entry point on the skin of the subject 505 and rotates the instrument 310 with the instrument holder 100 to line up with the entry point. Then the practitioner inserts the instrument 310 about 3 mm into the skin of the subject 505 to establish a pivot point. Alternatively, the practitioner may stop the instrument 310 right above the entry point on the skin of the subject 505 and only insert the instrument at a later stage.

As shown in FIG. 5E the practitioner returns the imaging system 520 to the a degree position to verify that the instrument 310 is pointing toward the target. Optionally, the instrument 310 or its virtual extension may be identified as not passing through the target when viewed with the imaging system 520 from the a angle.

As shown in FIG. 5F the positioning arm 510 is moved forward or back to tilt instrument holder 100 and instrument 310 so that instrument 310 will be aligned with the target 560 or so that the instrument's extension will be aligned with the target 560.

As shown in FIG. 5G once the instrument 310 is aligned in 3D to the target the practitioner can view the display 550 and gently push the instrument 310 toward the target 560 until the instrument 310 hits the target 560. Optionally, the instrument can be used to break up the target and/or vacuum it out from within the patient.

FIGS. 6A to 6F are schematic illustrations of a first alternative process of aligning instrument holder 200, according to an exemplary embodiment of the disclosure. In an exemplary embodiment of the disclosure, as shown in FIG. 6A, the subject 505 is positioned on a surgical table 530 with instrument holder 200 mounted on positioning arm 510 above the subject 505.

The positioning arm 510 is typically attached to the surgical table 530 and an imaging system 520 is initially positioned at an angle α relative to the vertical. With the help of imaging system 520 the practitioner views the display 550 and selects an angled plane relative to α (for example at an angle X), which will enable optimal insertion of the instrument 310 to hit the target 560 (e.g. a kidney stone). Additionally, the practitioner selects a puncture angle for tilting the instrument 310 in the selected angled plane to accurately hit the target 560.

In an exemplary embodiment of the disclosure, the practitioner rotates the imaging system 520 to the desired angle (X+ α) and views instrument holder 200 as appearing on display 550. Alternatively, if instrument holder 400 is mounted on positioning arm 510, the practitioner rotates the imaging system 520 to the desired angle (X+ α), inserts instrument 310 into instrument holder 400 and views instrument holder 400 and instrument 310 as appearing on display 550.

The practitioner takes a snapshot with the imaging system 520 that clearly shows the target 560, the first layer marking 205 of instrument holder 200, and the second layer marking 215 of instrument holder 200. Alternatively, if instrument holder 400 is used, the practitioner takes a snapshot with the imaging system 520 that shows the target 560, the first layer marking 405 of instrument holder 400, and the instrument 310 within instrument holder 400.

The practitioner reads the deviation of the second layer marking 215 of instrument holder 200 from the middle line of the first layer marking 205 of instrument holder 200 and the target 560. Alternatively, if instrument holder 400 is used, the practitioner reads the deviation of instrument 310 within instrument holder 400 from the middle line of the first layer marking 405 of instrument holder 400.

As shown in FIG. 6B, the practitioner may use the deviation as a guide to move the positioning arm 510 to align the second layer marking 215 of instrument holder 200 to point toward target 560 and to be in the center of first layer marking 205. Optionally, second layer marking 215 is in the form of a line and first layer marking 205 is in the form of lines representing linear graduations of a unit of measure (e.g. centimeter or inch). When instrument holder 200 is aligned with the selected plane the line of the second layer marking 215 will be centered on the middle line of the first layer marking 205.

Alternatively, if instrument holder 400 is used, the practitioner uses the deviation as guide to move the positioning arm to align the instrument 310 within instrument holder 400 to point toward the target and to be in the center of first layer marking 405. Optionally, first layer marking 405 is in the form of lines representing linear graduations of a unit of measure (e.g. centimeter or inch). Optionally, the first layer marking 405 also includes one or more pairs of arrows. When instrument holder 400 is aligned with the selected plane the instrument 310 within instrument holder 400 will be centered on the middle line of the first layer marking 405.

As shown in FIG. 6C the practitioner inserts the instrument 310 (e.g. a hollow needle) into the instrument holder 200, decides on an entry point on the skin of the subject 505 and rotates the instrument 310 with the instrument holder 200 to line up with the entry point. Then the practitioner inserts the instrument 310 about 3 mm into the skin of the subject 505 to establish a pivot point. Alternatively, the practitioner may stop the instrument 310 right above the entry point on the skin of the subject 505 and only insert the instrument 310 at a later stage.

As shown in FIG. 6D the practitioner returns the imaging system 520 to the a degree position to verify that the instrument 310 is pointing toward the target. Optionally, the instrument 310 or its virtual extension may be identified as not passing through the target when viewed with the imaging system 520 from the a angle.

As shown in FIG. 6E the positioning arm 510 is moved forward or back to tilt instrument holder 200 and instrument 310 so that the instrument 310 will be aligned with the target 560 or so that the instrument's extension will be aligned with the target 560.

As shown in FIG. 6F once the instrument 310 is aligned in 3D to the target the practitioner can view the display 550 and gently push the instrument 310 toward the target 560 until the instrument 310 hits the target 560. Optionally, the instrument can be used to break up the target and/or vacuum it out from within the patient.

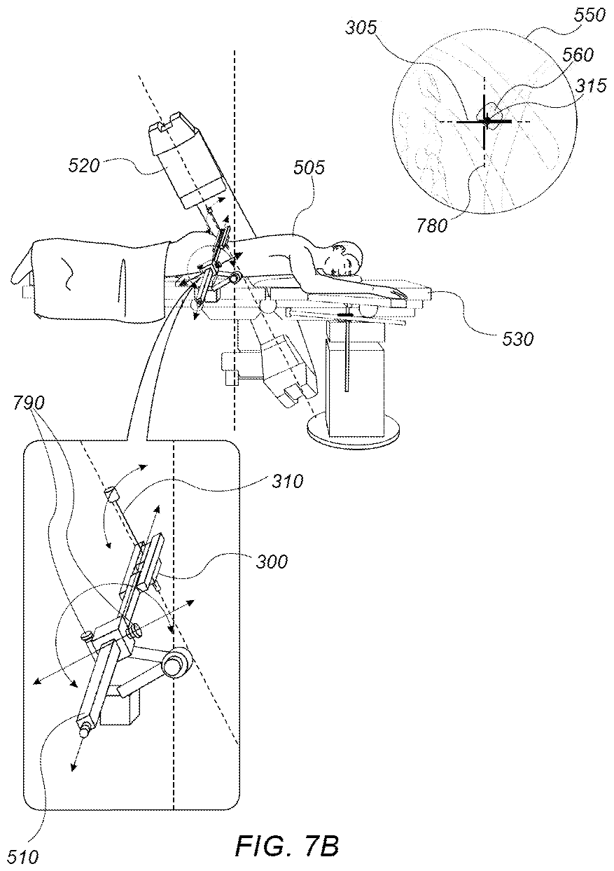

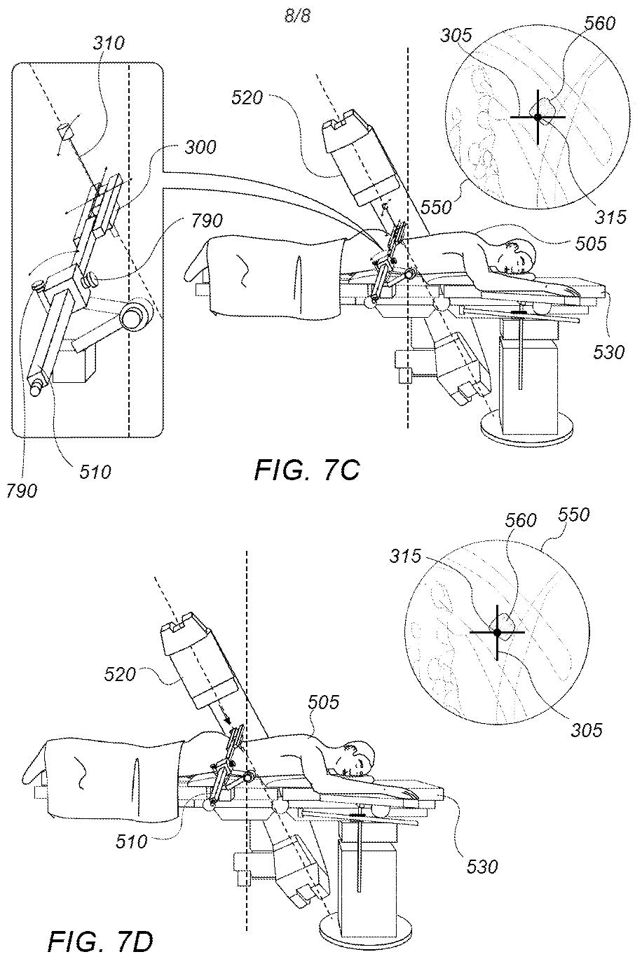

FIGS. 7A to 7D are schematic illustrations of a second alternative process of aligning a second alternative instrument holder 300, according to an exemplary embodiment of the disclosure. In an exemplary embodiment of the disclosure, the practitioner selects a puncture angle, an insertion point 780 and marks the insertion point 780 on the skin of the subject 505. FIG. 7A shows a coordinate system 770 with axis relative to the insertion point 780. The axis including the cranial direction (head end), caudal direction (rear end), the medial direction (center of the subject) and the lateral direction (outer end relative to the entry point on the subject). In an exemplary embodiment of the disclosure, the practitioner rotates the imaging system 520 in both the median plane (cranial-caudal) and the transverse plane (medial-lateral) to line up with the insertion point 780 and the target 560 in the display 550. The practitioner further deploys the positioning arm 510 with instrument holder 300 mounted thereon to align the instrument 310 toward the target 560. The positioning arm 510 is designed with multiple degrees of freedom so that the instrument holder 300 may be oriented in any direction. Likewise, the articulated arm includes various locking knobs 790 to lock elements of the positioning arm 510 and prevent them from moving before pushing instrument 310 into the body of the subject 505.

As shown in FIG. 7B the practitioner moves the positioning arm 510 to align the first layer marking 305 of instrument holder 300 with the second layer marking 315, with the marked insertion point 780 and the target 560 as accurately as possible.

As shown in FIG. 7C the practitioner may use features of positioning arm 510 to fine tune the positioning of the instrument 310 relative to target 560 and then lock positioning arm 510 so that the instrument 310 is held steady and won't be misaligned when pushing the instrument 310 into the body of the subject 505 via the instrument holder 300.

As shown in FIG. 7D, once the instrument holder 300 and instrument 310 are aligned the practitioner can push the instrument 310 to percutaneously reach the target and deal with it. For example, extract a kidney stone or deliver medication.

It should be appreciated that the above described methods and apparatus may be varied in many ways, including omitting or adding steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by persons skilled in the art that the present invention is not limited to what has been particularly shown and described hereinabove. Rather the scope of the present invention is defined only by the claims, which follow.

1. An instrument holder for aligning an instrument for accessing a target within a subject, comprising:
a first layer;
a second layer;
an instrument guide having an entry point and an exit point, the instrument guide connecting between the first layer and the second layer and configured to guide the instrument from the entry point to the exit point;
wherein the first layer and the second layer include first layer markings and second layer markings that are visible by an imaging system; and
wherein the first layer markings are designed to form a predefined pattern with the second layer markings relative to the target as viewed on a display of the imaging system, when aligning the first layer and the second layer so that the instrument will collide with the target.

2. The instrument holder according to claim 1, wherein the first layer markings include a first geometric shape and the second layer markings include a second geometric shape distinct from the first geometric shape.

3. The instrument holder according to claim 2, wherein the first geometric shape comprises a line.

4. The instrument holder according to claim 3, wherein the second geometric shape comprises one or more pairs of arrows or arrow heads, and in the predefined pattern the one or more pairs of arrows or arrow heads point at the line, which is situated between the one or more pairs of arrows or arrow heads.

5. The instrument holder according to claim 4, wherein in the predefined pattern, there is a gap between the one or more pairs of arrows or arrow heads and the line.

6. The instrument holder according to claim 2, wherein the first geometric shape comprises lines representing linear graduations of a unit of measure.

7. The instrument holder according to claim 6, wherein the second geometric shape comprises a line, and in the predefined pattern the line of the second geometric shape aligns at a middle line of the first geometric shape.

8. The instrument holder according to claim 2, wherein the first geometric shape comprises one or more pairs of arrows or arrowheads.

9. The instrument holder according to claim 8, wherein the second geometric shape comprises two lines in the shape of a cross with the exit point at the center, and in the predefined pattern the one or more pairs of arrows or arrow heads point at the lines of the second geometric shape such that each line is situated between the one or more pairs of arrows or arrow heads.

10. The instrument holder according to claim 9, wherein in the predefined pattern, there is a gap between the one or more pairs of arrows or arrow heads and the lines of the second geometric shape.

11. The instrument holder according to claim 2, wherein the first geometric shape comprises two or more sets of lines representing linear graduations of a unit of measure.

12. The instrument holder according to claim 11, wherein the second geometric shape comprises two lines in the shape of a cross with the exit point at the center, and in the predefined pattern each of the lines of the second geometric shape aligns with a middle line of each of the sets of lines representing linear graduations of a unit of measure.

13. The instrument holder according to claim 1, wherein the first layer markings include a cross with the entry point at the center and the second layer markings include a cross with the exit point at the center, and in the predefined pattern the cross of the first layer marking and the cross of the second layer marking coincide.

14. The instrument holder according to claim 1, further comprising a protuberance extending from the second layer.

15. The instrument holder according to claim 1, wherein the instrument is a hollow needle.

16. The instrument holder according to claim 1, wherein the instrument is an elongated tool.

17. The instrument holder according to claim 1, wherein the instrument holder includes handles to fit a positioning arm.

18. The instrument holder according to claim 17, wherein the handles are configured to enable the instrument holder to rotate relative to the positioning arm.

19. The instrument holder according to claim 1, wherein the first layer or the second layer include additional markings, which are not part of the predefined pattern to identify the first layer or the second layer by a user when aligning.

20. The instrument holder according to claim 1, wherein the target is located percutaneously.

21. The instrument holder according to claim 1, wherein the instrument holder is transparent to the imaging system.

22. The instrument holder according to claim 1, wherein the instrument guide is perpendicular to the first layer and the second layer.

23. The instrument holder according to claim 1, wherein the instrument guide forms a preselected angle with the first layer and with the second layer.

24. A method for aligning an instrument to a target within a subject, comprising:
providing an instrument holder as in claim 1 on a positioning arm;
positioning an imaging system to show on a display the target and the instrument holder; and
aligning the instrument holder so that the first layer markings and the second layer markings form a predefined pattern relative to the target on the display of the imaging system.

25. The method according to claim 24, wherein the instrument is being aligned for percutaneous access to the target within the subject.

26. The method according to claim 24, wherein the positioning arm is an articulated arm positioned above the subject.

27. An instrument holder for aligning an instrument for accessing a target within a subject, comprising:
a first layer;
a second layer, the second layer connected to the first layer by a column;
an instrument guide having an entry point and an exit point, the instrument guide located within the second layer and configured to guide the instrument from the entry point to the exit point;
wherein the first layer includes markings forming a first layer markings that are visible by an imaging system; and
wherein the first layer markings are designed to form a predefined pattern with the instrument visible by the imaging system within the second layer relative to the target as viewed on a display of the imaging system, when aligning the first layer and the second layer so that the instrument will collide with the target.

28. The instrument holder according to claim 27, wherein the first layer markings further comprise a first geometric shape.

29. The instrument holder according to claim 28, wherein the first geometric shape comprises lines representing linear graduations of a unit of measure and in the predefined pattern the instrument aligns at a middle line of the first geometric shape.

30. The instrument holder according to claim 28, wherein the first geometric shape comprises one or more pairs of arrows or arrow heads, and in the predefined pattern the one or more pairs of arrows or arrow heads point at the instrument, which is situated between the one or more pairs of arrows or arrow heads.

31. The instrument holder according to claim 30, wherein there is a gap between the one or more pairs of arrows or arrow heads and the instrument.

32. The instrument holder according to claim 27, further comprising a protuberance extending from the second layer.

33. The instrument holder according to claim 27, wherein the instrument is a hollow needle.

34. The instrument holder according to claim 27, wherein the instrument is an elongated tool.

35. The instrument holder according to claim 27, wherein the instrument holder includes handles to fit a positioning arm.

36. The instrument holder according to claim 35, wherein the handles are configured to enable the instrument holder to rotate relative to the positioning arm.

37. The instrument holder according to claim 27, wherein the first layer or the second layer include additional markings, which are not part of the predefined pattern to identify the first layer or the second layer by a user when aligning.

38. The instrument holder according to claim 27, wherein the target is located percutaneously.

39. The instrument holder according to claim 27, wherein the instrument holder is transparent to the imaging system.

40. A method for aligning an instrument to a target within a subject, comprising:
providing the instrument holder of claim 27 on a positioning arm;
inserting the instrument into the instrument holder;
positioning an imaging system to show on a display the target, the instrument holder and the instrument; and
aligning the instrument holder so that the first layer markings and the instrument form a predefined pattern relative to the target on the display of the imaging system.

41. The method according to claim 40, wherein the instrument is being aligned for percutaneous access to the target within the subject.

42. The method according to claim 40, wherein the positioning arm is an articulated arm positioned above the subject.
